(19) United States
(12) Patent Application Publication
(10) Pub. No.: US 2026/0069522 A1
(43) Pub. Date: Mar. 12, 2026

(54) SKIN DEPIGMENTATION COMPOSITION AND USE THEREOF

(71) Applicants: Behrooz KASRAEE, Chatelaine (CH); Laure DIRLEWANGER, Arzier-Le Muids (CH)

(72) Inventors: Behrooz KASRAEE, Chatelaine (CH); Laure DIRLEWANGER, Arzier-Le Muids (CH)

(21) Appl. No.: 19/108,789

(22) PCT Filed: Sep. 11, 2023
(86) PCT No.: PCT/EP2023/074833

§ 371 Date: Mar. 5, 2025

(30) Foreign Application Priority Data
Sep. 12, 2022 (EP) 22195129.6

(51) Int. Cl. A61K 8/46; A61K 8/365; A61K 8/42; A61Q 19/02

(52) U.S. Cl. CPC A61K 8/46; A61K 8/365; A61K 8/42; A61Q 19/02

(57) ABSTRACT

The present invention relates to a composition comprising cysteamine, azabenzene-4-carboxamide and glycolic acid. The present invention also relates to the cosmetic use thereof for inhibiting, reducing or preventing skin pigmentation and to the therapeutical use thereof for inhibiting, reducing or preventing skin hyperpigmentation diseases or disorders.

### FIELD OF THE INVENTION

The present invention relates to a composition comprising cysteamine, azabenzene-4-carboxamide and glycolic acid. The present invention also relates to the cosmetic use thereof for inhibiting, reducing or preventing skin pigmentation and to the therapeutical use thereof for inhibiting, reducing or preventing skin hyperpigmentation diseases or disorders. The composition of the invention provides reduced irritation of the skin, increases the long-term depigmenting efficacy after 8 weeks of treatment, provides immediate depigmenting effect, has increased shelf-life and reduced unpleasant odour.

### BACKGROUND OF THE INVENTION

Human skin colours are quite variable around the world. Cutaneous coloration in humans arises from a complex series of cellular processes that are carried out within the melanocytes located in the lower part of the epidermis. These processes result in the synthesis and transfer of a pigment, melanin, which, besides being responsible for skin colour and tone, is the key physiological defence against sun-induced damage, such as sunburn, photoaging and photocarcinogenesis.

In an effort to simply obtain brighter/lighter skin or address the hyperpigmentation disorders, such as melasma, freckles, lentigos, and skin dark spots, various compositions have been formulated. The use of such compositions is not limited for use in treating pigmentation diseases or disorders but is also used in some cultures/markets merely for the purpose of changing or modifying one's natural healthy skin colour.

A large number of agents and methods for skin depigmentation have been developed and put on the market. Such methods include the oral administration of large doses of Vitamin C, the parenteral administration of glutathione, the topical administration of peroxide bleaching agents such as hydrogen peroxide for skin depigmentation, zinc peroxide, sodium peroxide and the like, and the topical application of Vitamin C and/or cysteine. Vitamin C, however, has stability issues, especially in water-based formulations, resulting in colour and odour changes. Thiol compounds such as glutathione and cysteine have slow and/or generally poor depigmentation performance properties.

The most commonly employed depigmenting agent has been hydroquinone and its derivatives. However, these compounds, while effective, have serious, detrimental side effects. Even at concentrations below 2%, hydroquinone is both irritating and cytotoxic to the melanocytes. Similar problems have been experienced with peroxide depigmenting agents as well. Another known depigmenting agent is tretinoin, an effective treatment for both wrinkles and skin pigmentation but is also known to cause skin irritation that can lead to skin darkening.

A wide range of polyphenols present in plant extracts have also been used for skin depigmentation purposes. Such natural polyphenols are for example anthraquinones, arylbenzofurans, chalcones, coumarins, and flavonoids. One class of polyphenols compounds that has received a lot of attention is that based on substituted resorcinols and their derivatives. However, despite their relatively good skin lightening capabilities, they tend to suffer from stability issues, which also oftentimes coincide with loss of skin lightening efficacy, rendering them generally unsuitable for topical applications.

Another agent, which demonstrated interesting depigmenting effects is nicotinamide. Nicotinamide is a 3-substituted pyridine which exerts its skin depigmenting effect through the inhibition of melanosomal transfer from melanocytes to keratinocytes. Despite good tolerability on human skin, nicotinamide has a poor skin depigmenting efficacy.

Cysteamine is incorporated as a therapeutic agent in a variety of topical and dermatological compositions, such as pharmaceutical or cosmetic compositions, that are commonly used as drugs in managing skin depigmentation. However, the unpleasant odour leads to poor patient compliance and limited use thereof.

Cysteamine has also been combined with citric acid, glycolic acid or lactic acid (Swiss Patent No CH 706 226 A2). However, this combination was related to citric acid, glycolic acid and lactic acid concentrations equal or less than 1%. The effect of higher concentrations of citric acid, glycolic acid and lactic acid in combination with cysteamine is not reported in the prior art, probably due to risks of inflammation which is associated with higher concentrations of organic acids.

Consequently, there is still a need for a skin depigmentation composition that provides effective lightening capabilities in just few weeks and does not cause significant inflammation, irritation, or photosensitivity of the skin following application.

The ideal skin depigmentation composition should have a potent, rapid and selective depigmentation effect, carry no short- or long-term side-effects, be well tolerated, act at one or more steps of the pigmentation process, have sufficiently long shelf-life, and for which the odour should not be perceived as unpleasant.

### SUMMARY OF THE INVENTION

An aspect of the present invention provides a composition comprising
0.2% to 20% w/w of cysteamine or a salt thereof,
0.2% to 20% w/w of azabenzene-4-carboxamide or a salt thereof, and
2% to 20% w/w of glycolic acid.

Another aspect of the present invention provides a composition of the invention for use in a method for inhibiting, reducing or preventing skin hyperpigmentation diseases or disorders in a subject.

A further aspect of the present invention provides a cosmetical method for inhibiting, reducing or preventing pigmentation of normal healthy skin, comprising topically applying the composition of the invention to the skin of a subject in need thereof.

A further aspect of the present invention provides a cosmetical method for inhibiting, reducing or preventing pigmentation of normal healthy skin, comprising the steps of
a) topically applying to an area of the skin of a subject in need thereof a first composition comprising
0.2% to 20% w/w of cysteamine or a salt thereof, and
2% to 20% w/w of glycolic acid,
b) rinsing off the first composition,
c) topically applying to said area of the skin a second composition comprising 0.2% to 20% w/w of an azabenzene-4-carboxamide or a salt thereof,
d) repeating the steps a), b) and c) to obtain desired skin depigmentation effect.

Another aspect of the present invention provides a combination product comprising a first composition and a second composition for separate topical application, wherein
the first composition comprising 0.2% to 20% w/w of cysteamine or a salt thereof and 2% to 20% w/w of glycolic acid, and
the second composition comprising 0.2% to 20% w/w of azabenzene-4-carboxamide or a salt thereof.

Another aspect of the present invention provides a method of reducing the unpleasant odour of cysteamine or a salt thereof, comprising combining 0.2% to 20% w/w cysteamine or a salt thereof with 0.2% to 20% w/w azabenzene-4-carboxamide or a salt thereof and 2% to 20% w/w glycolic acid.

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”.

As used in the specification and claims, the singular form “a”, “an” and “the” include plural references unless the context clearly dictates otherwise.

As used in the specification and claims, the term “and/or” used in a phrase such as “A and/or B” herein is intended to include “A and B”, “A or B”, “A”, and “B”.

As used herein, the term “at least one” means “one or more” and also encompasses the terms “at least two”, “at least three”, “at least four”, etc.

As used herein the term “therapeutically effective amount” is defined as an amount of the agent that is sufficient to detectably and repeatedly ameliorate, reduce, minimize or limit the extent of the skin disorder, disease or condition or its symptoms.

As used herein the term “depigmentation effective amount” means an amount of a compound or composition sufficient to significantly induce a positive benefit on skin depigmentation, preferably a reduction in the melanization or rate of melanization of the skin, but low enough to avoid serious side effects.

As used herein the term “pharmaceutically acceptable excipient” means that the compositions or components thereof so described are suitable for suitable for administration to subject (patient) body without undue toxicity, incompatibility, instability, irritability, allergic response, and the like.

As used herein the term “dermatologically acceptable” means that the compositions or components thereof so described are suitable for use in contact with skin of a mammal, preferably of human, without undue toxicity, incompatibility, instability, irritability, allergic response, and the like.

As used herein, the term “treatment” refers to a therapeutic or preventative measure. The treatment may be administered to a subject having a medical disorder, such as skin disorder or condition, or who ultimately may acquire the disorder or condition, in order to prevent, cure, delay, reduce the severity of, or ameliorate one or more symptoms of a disorder or recurring disorder.

As used herein, the term “cosmetic”, “cosmetic management” or “cosmetic treatment” is non-therapeutic treatment of skin conditions that are not diseases or pathological states.

As used herein the term “topical” or “topically” refers to the application of the composition of the present invention onto the surface of the skin and/or a portion thereof, such as facial skin, skin on the neck, skin on the arms, skin on the hands, skin on the legs and skin on the scalp, skin on the elbows, skin on the knees, skin on the male or female genital and anus areas so that it can display local activity. Accordingly, the topical forms embrace cosmetical, dermatological and/or pharmaceutical dosage forms which are suitable for external use, so that a direct contact with the skin and/or a portion thereof results.

As used herein the terms “subject” are well-recognized in the art, and, are used herein to refer to a mammal, and most preferably a human. In some embodiments, the subject is a subject in need of treatment or a subject with a skin pigmentation disease or disorder, such as hyperpigmentation. However, in other embodiments, the subject can be a normal subject who has a normal healthy skin and who needs to lighten (whiten) his skin. The term does not denote a particular age or sex. Thus, adult, young and newborn subjects, whether male or female, are intended to be covered.

As used herein the term “depigmentation” (or lightening, bleaching, whitening and brightening used interchangeably herein) is the lightening of the skin, or loss of pigment. The skin depigmentation agents or compositions are also referred as “skin lightener”, “skin whitener”, “skin even-toner” and “skin brightener”. Whatever terminology is employed, the general premise is that they all relate to a reduction in the melanization or rate of melanization of the skin, which results in loss of pigment.

As used herein the term “immediate depigmentation effect”, “immediate depigmenting effect” or “immediate skin depigmentation” is a visual effect leading to a lighter skin tone within seconds or few minutes after application of the topical product/composition. This effect leads to immediate skin radiance and more even skin tone and it is perceived by a reduction of the melanin index.

As used herein the term “post-inflammatory hyperpigmentation” refers to the changes in melanin content as a response to an inflammatory event (e.g., acne, scratch, laser therapy, insect sting or bite, sunburn, etc), especially in individuals of darker skin tone or colour.

As used herein the term “reduction” or “reducing” the unpleasant odour, such as sulphur odour, of cysteamine or a salt or an ester thereof, refers to attenuation, lessening and/or diminution of the unpleasant odour so that the unpleasant odour is no more perceptible or almost not perceptible by a human subject. It refers also to elimination and/or disappearance of the unpleasant odour.

The Applicant has surprisingly found that a combination of 0.2% to 20% w/w of cysteamine or a salt thereof, 0.2% to 20% w/w of azabenzene-4-carboxamide or a salt thereof, and 2% to 20% w/w of glycolic acid provides unexpected benefits and synergistic effects in the following fields: long term and immediate skin depigmentation, anti-inflammatory effect, increase of the shelf-life and reduction of the unpleasant odour of cysteamine or a salt thereof.

Firstly, the Applicant has surprisingly found that a combination of 0.2% to 20% w/w of cysteamine or a salt thereof, 0.2% to 20% w/w of azabenzene-4-carboxamide or a salt thereof, and 2% to 20% w/w of glycolic acid, provides a less irritant formulation compared to those of the prior art comprising less concentration of glycolic acid, while glycolic acid itself is considered as an irritant substance in medical literature.

Secondly, contrary to existing cysteamine formulations that contain glycolic acid at a concentration of less than 1%, the composition of the present invention comprising cysteamine, azabenzene-4-carboxamide and 2% or more glycolic acid exerts an immediate skin depigmenting effect after only few seconds of application to human skin, while having a reduced unpleasant odour of cysteamine. An immediate depigmenting effect is very rare and adds a huge value and benefit to the product (composition), especially in term of patient's compliance. Indeed, one of the most frequent issues with depigmenting topical therapies is their onset of action: it usually takes 2 to 4 weeks to see the very first results. Thus many patients stop the treatment earlier by lack of motivation because no depigmenting effect is seen after the first applications. Such immediate depigmenting effect does not happen with combination of cysteamine and azabenzene-4-carboxamide associated with other alpha-hydroxy acids (AHAs), such as lactic acid or tartaric acid.

Thirdly, the Applicant has surprisingly found that the addition of azabenzene-4-carboxamide, even at low percentages, starting from 0.2% and up to 20%, into a composition comprising cysteamine or a salt thereof and glycolic acid, is increasing synergistically the long-term skin depigmenting effect and makes it significantly more visible and perceptible by the treated subjects, after 8 weeks of treatment. According to an embodiment, the Applicant has surprisingly found that the skin depigmenting effect after 8 weeks is even further improved, when azabenzene-4-carboxamide is not applied simultaneously with cysteamine or a salt thereof and glycolic acid, but in a method of skin depigmentation (“2-step” method).

Furthermore, the Applicant has surprisingly found that the addition of 0.2% to 20% w/w of azabenzene-4-carboxamide or a salt thereof into a composition already comprising cysteamine or a salt thereof and glycolic acid provides an even bigger reduction of the unpleasant odour of cysteamine, leading to the increase of the acceptability of the product by the treated subjects.

Lastly, the combination of cysteamine with glycolic acid at concentration equal or superior to 2% together with azabenzene-4-carboxamide exerts a surprising synergistic effect for the increase of shelf-life and durability of the product, which is to date not known.

Thus an aspect of the present invention provides a composition comprising
0.2% to 20% w/w of cysteamine or a salt thereof,
0.2% to 20% w/w of azabenzene-4-carboxamide or a salt thereof, and
2% to 20% w/w of glycolic acid.

In some embodiments, the compositions of the invention comprise 5% to 20% w/w of cysteamine or a salt thereof, or 2% to 12% w/w of cysteamine or a salt thereof, or 5% to 12% w/w of cysteamine or a salt thereof, or 0.2% to 7% w/w of cysteamine of a salt thereof, or 2% to 7% w/w of cysteamine or a salt thereof, or 3% to 20% w/w of cysteamine or a salt thereof, or 1% to 12% w/w of cysteamine or a salt thereof, or 3% to 12% w/w of cysteamine or a salt thereof, or 0.2% to 7% w/w of cysteamine of a salt thereof, or 1% to 7% w/w of cysteamine or a salt thereof.

In other embodiments, the compositions of the invention comprise 3% to 20% w/w of azabenzene-4-carboxamide or a salt thereof, or 2% to 10% w/w azabenzene-4-carboxamide or a salt thereof, or 3% to 10% w/w of azabenzene-4-carboxamide or a salt thereof, or 0.2% to 10% w/w of azabenzene-4-carboxamide or a salt thereof, or 0.5% to 10% w/w of azabenzene-4-carboxamide or a salt thereof.

In further embodiments, the compositions of the invention comprise 3.5% to 20% w/w of glycolic acid, or 3.5% to 15% w/w of glycolic acid, or 2% to 10% w/w of glycolic acid, or 2% to 15% w/w of glycolic acid.

In an embodiment, the composition of the invention comprises
5% to 20% w/w of cysteamine or a salt thereof,
3% to 20% w/w of azabenzene-4-carboxamide or a salt thereof, and
3.5% to 20% w/w of glycolic acid.

In a further embodiment, the composition of the invention comprises
0.2% to 7% w/w of cysteamine or a salt thereof,
0.2% to 10% w/w of azabenzene-4-carboxamide or a salt thereof, and
2% to 10% w/w of glycolic acid.

The present invention provides also a combination product comprising or consisting of a first composition and a second composition for separate topical application, wherein
the first composition comprising 0.2% to 20% w/w of cysteamine or a salt thereof and 2% to 20% w/w of glycolic acid,
   and
the second composition comprising 0.2% to 20% w/w of azabenzene-4-carboxamide or a salt thereof.

In an embodiment, the combination product of the invention consists of
the first composition consisting of
   0.2% to 20% w/w of cysteamine or a salt thereof, preferably 5% to 20% w/w of cysteamine or a salt thereof, or 2% to 12% w/w of cysteamine or a salt thereof, or 5% to 12% w/w of cysteamine or a salt thereof, or 0.2% to 7% w/w of cysteamine of a salt thereof, or 2% to 7% w/w of cysteamine or a salt thereof, or 3% to 20% w/w of cysteamine or a salt thereof, or 1% to 12% w/w of cysteamine or a salt thereof, or 3% to 12% w/w of cysteamine or a salt thereof, or 0.2% to 7% w/w of cysteamine of a salt thereof, or 1% to 7% w/w of cysteamine or a salt thereof,
   2% to 20% w/w of glycolic acid, preferably 3.5% to 20% w/w of glycolic acid, or 3.5% to 15% w/w of glycolic acid, or 2% to 10% w/w of glycolic acid, or 2% to 15% w/w of glycolic acid,
   0.01% to 1% w/w retinol, and
   cosmetically acceptable excipients,
      and
the second composition consisting of
   0.2% to 20% w/w of azabenzene-4-carboxamide or a salt thereof, preferably 3% to 20% w/w of azabenzene-4-carboxamide or a salt thereof, or 2% to 10% w/w azabenzene-4-carboxamide or a salt thereof, or 3% to 10% w/w of azabenzene-4-carboxamide or a salt thereof, or 0.2% to 10% w/w of azabenzene-4-carboxamide or a salt thereof, or 0.5% to 10% w/w of azabenzene-4-carboxamide or a salt thereof,
   maximum 15% w/w of skin conditioning agents selected from xylitylglucoside, anhydroxylitol, xylitol, panthenol, Tazman pepper natural extract and/or combination thereof, preferably 0.01% to 15% w/w or 0.01% to 10% of skin conditioning agents selected from xylitylglucoside, anhydroxylitol, xylitol, panthenol, Tazman pepper natural extract and/or combination thereof, preferably 0.01% to 15% or 0.01% to 10% of skin conditioning agents selected from xylitylglucoside, anhydroxylitol, xylitol, panthenol, and/or combination thereof,
   0.01% to 1% retinol, and
   cosmetically acceptable excipients.

In the context of the present invention, Tazman pepper is a natural Australian active ingredient extracted from the Tasmanian pepper berry.

In some embodiments, the first composition of the combination product of the invention comprises 5% to 20% w/w of cysteamine or a salt thereof, or 2% to 12% w/w of cysteamine or a salt thereof, or 5% to 12% w/w of cysteamine or a salt thereof, or 0.2% to 7% w/w of cysteamine of a salt thereof, or 2% to 7% w/w of cysteamine or a salt thereof or 3% to 20% w/w of cysteamine or a salt thereof, or 1% to 12% w/w of cysteamine or a salt thereof, or 3% to 12% w/w of cysteamine or a salt thereof, or 0.2% to 7% w/w of cysteamine of a salt thereof, or 1% to 7% w/w of cysteamine or a salt thereof.

In other embodiments, the first composition of the combination product of the invention comprises 3.5% to 20% w/w of glycolic acid, or 3.5% to 15% w/w of glycolic acid, or 2% to 10% w/w of glycolic acid, or 2% to 15% w/w of glycolic acid.

In further embodiments, the second composition of the combination product of the invention comprises 3% to 20% w/w of azabenzene-4-carboxamide or a salt thereof, or 2% to 10% w/w azabenzene-4-carboxamide or a salt thereof, or 3% to 10% w/w of azabenzene-4-carboxamide or a salt thereof, or 0.2% to 10% w/w of azabenzene-4-carboxamide or a salt thereof, or 0.5% to 10% w/w of azabenzene-4-carboxamide or a salt thereof.

In the present disclosure, the % w/w of cysteamine or a salt thereof refers to % w/w of cysteamine (cysteamine free base). Thus reference to any particular mass of “cysteamine or a salt thereof” refers to the mass equivalent of the free base (cysteamine free base) and reference to any particular mass of a specific salt of cysteamine, such as cysteamine hydrochloride, refers to the mass of the specific salt. The above-mentioned definition can be also applied to % w/w of azabenzene-4-carboxamide or a salt thereof, for example reference to any particular mass of “azabenzene-4-carboxamide or a salt thereof” refers to the mass equivalent of the free base (azabenzene-4-carboxamide)

The term “cysteamine” is intended here to cover any (pharmaceutically or cosmetically or dermatologically) acceptable salt, ester, solvate, hydrate, prodrug, or any other compound which, upon administration to the patient is capable of providing (directly or indirectly) cysteamine. Preferred salts of cysteamine are hydrochloride, bitartrate, phosphate. Any compound that is a prodrug of cysteamine is within the scope and spirit of the invention. The term “prodrug” is used in its broadest sense and encompasses those derivatives that are converted in vivo to cysteamine. The prodrug can hydrolyse, oxidize, or otherwise react under biological conditions to provide cysteamine. Examples of prodrugs include, but are not limited to, derivatives and metabolites of cysteamine that include biohydrolysable moieties such as biohydrolysable amides, biohydrolysable esters, biohydrolysable carbamates, biohydrolysable carbonates, biohydrolysable ureides, and biohydrolysable phosphate analogues. Prodrugs can typically be prepared using well-known methods, such as those described by Burger in “Medicinal Chemistry and Drug Discovery” 6th ed. (Donald J. Abraham ed., 2001, Wiley) and “Design and Applications of Prodrugs” (H. Bundgaard ed., 1985, Harwood Academic Publishers). In addition, cysteamine referred to herein may be in crystalline or amorphous form either as free compounds or as solvates (e.g. hydrates) and it is intended that all forms are within the scope of the present invention. Methods of solvation are generally known within the art.

The salts of azabenzene-4-carboxamide are pharmaceutical acceptable salts, cosmetical acceptable salts, and dermatological acceptable salts.

The term “pharmaceutically acceptable salts” as used herein refers to salts that retain the desired biological activity of azabenzene-4-carboxamide and include pharmaceutically acceptable acid addition salts and base addition salts. Suitable pharmaceutically acceptable acid addition salts of azabenzene-4-carboxamide may be prepared from an inorganic acid or from an organic acid or can be prepared in situ during the final isolation and purification of azabenzene-4-carboxamide. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Suitable pharmaceutically acceptable base addition salts of azabenzene-4-carboxamide include metallic salts made from lithium, sodium, potassium, magnesium, calcium, aluminium, and zinc, and organic salts made from organic bases such as choline, diethanolamine, morpholine. Other examples of organic salts are ammonium salts, quaternary salts such as tetramethylammonium salt; amino acid addition salts such as salts with glycine and arginine. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention.

The combination products and the compositions of the present invention can be prepared by any method known in the art for cosmetic, dermatological and/or pharmaceutical compositions. Generally, the method comprises the simple mixing or blending of the components; though, especially where insoluble or immiscible components are employed higher agitation or homogenization may be necessary to prepare an appropriate composition, e.g., an emulsion or suspension, etc. Additionally, during the preparation, it may be desirable to add known pH adjusters, in order to maintain a proper pH of the composition for topical application, especially if basic ingredients are to be employed. Generally, the pH should range from about be 3.5 to about 8.

In some embodiments, the compositions of the invention are cosmetic compositions further comprising cosmetically acceptable excipients for topical administrations.

In some embodiments, the compositions of the inventions are dermatological compositions further comprising dermatologically acceptable excipients for dermatological administrations.

In some embodiments, the compositions of the inventions are pharmaceutical compositions further comprising pharmaceutically acceptable excipients for oral or topical administrations.

In some embodiments, the present invention provides a composition consisting of
0.2% to 20% w/w of cysteamine or a salt thereof,
0.2% to 20% w/w of azabenzene-4-carboxamide or a salt thereof,
2% to 20% w/w of glycolic acid,
0.01% to 1% w/w retinol, and
cosmetically acceptable excipients for topical administration or pharmaceutically acceptable excipients for oral or topical administration.

The cosmetic compositions, dermatological compositions and pharmaceutical compositions according to the invention further comprise one or more cosmetically, dermatologically or pharmaceutically acceptable excipients, as are used conventionally in such compositions, for example preservatives, antioxidants, chelating agents, bactericides, perfumes, substances for preventing foaming, dyestuffs, pigments which have a colouring effect, thickeners, propellants, surfactant substances, emulsifiers, softening, moisturizing and/or moisture-retaining substances, distilled water, fats, oils, waxes or other conventional constituents of cosmetic, dermatological or pharmaceutical compositions, such as alcohols, polyols, polymers, foam stabilizers, electrolytes, organic solvents or silicone derivatives. The necessary amounts of the cosmetically, dermatologically or pharmaceutically acceptable excipients can, based on the desired product, easily be chosen by a person skilled in the art.

A moisturizing substance may be incorporated into compositions of the invention to maintain hydration or rehydrate the skin. Moisturizers that prevent water from evaporating from the skin by providing a protective coating are called emollients. Additionally, an emollient provides a softening or soothing effect on the skin surface and is generally considered safe for topical use. Preferred emollients include mineral oils, lanolin, petrolatum, capric/caprylic triglyceraldehydes, cholesterol, silicones such as dimeticone, cyclometicone, almond oil, jojoba oil, avocado oil, castor oil, sesame oil, sunflower oil, coconut oil and grape seed oil, cocoa butter, olive oil, aloe extracts, fatty acids such as oleic and stearic, fatty alcohols such as cetyl and hexadecyl (ENJAY), diisopropyl adipate, hydroxybenzoate esters, benzoic acid esters of C₉₋₁₅-alcohols, isonononyl isononanoate, ethers such as polyoxypropylene butyl ethers and polyoxypropylene cetyl ethers, and C₁₂₋₁₅-alkyl benzoates, and mixtures thereof. Moisturizing substances that bind water, thereby retaining it on the skin surface are called humectants. Suitable humectants can be incorporated into the skin depigmenting compositions of the present invention such as glycerin, polypropylene glycol, polyethylene glycol, lactic acid, pyrrolidone carboxylic acid, urea, phospholipids, collagen, elastin, ceramides, lecithin sorbitol, PEG-4, and mixtures thereof.

The compositions of the invention can also contain the usual alcohols, especially lower alcohols, preferably ethanol and/or isopropanol, low diols or polyols and their ethers, preferably propyleneglycol, glycerin, ethyleneglycol, ethyleneglycol monoethyl- or monobutylether, propyleneglycol monomethyl- or monoethyl- or -monobutylether, diethyleneglycol monomethyl- or monoethylether and analogue products, polymers, foam stabilizers; electrolytes and especially one or more thickeners. Thickeners that may be used in the compositions of the invention to assist in making the consistency of a product suitable include carbomer, silicium dioxide, magnesium and/or aluminium silicates, beeswax, stearic acid, stearyl alcohol polysaccharides and their derivatives such as xanthan gum, hydroxypropyl cellulose, polyacrylamides, acrylate crosspolymers preferably a carbomer, such as Carbopol® of type 980, 981, 1382, 2984, 5984 alone or mixtures thereof.

Suitable stabilizing agents which may be included in the compositions of the invention to stabilize components such as e.g. an emulsifier or a foam builder/stabilizer include but are not limited to alkali hydroxides such as a sodium and potassium hydroxide; organic bases such as diethanolamine (DEA), triethanolamine (TEA), aminomethyl propanol, and mixtures thereof, amino acids such as arginine and lysine and any combination of any foregoing.

The compositions of the present invention may also contain filter substances that absorb UV radiation, or sunscreens, wherein the total quantity of filter substances is, for example 0.001 to 30%, preferably 0.5 to 10%, based on the total weight of the preparation. The compositions may also serve as sunscreen agents for the skin. Such UV filter substances include, for example, the following: avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

The compositions of the invention may also include one or more skin penetrants. These are additives that, when applied to the skin, have a direct effect on the permeability of the skin barrier: increasing the speed with which and/or the amount by which certain other compounds are able to penetrate into the skin layers. Exemplary organic penetration enhancers include dimethyl sulfoxide; isopropyl myristate; decyl, undecyl or dodecyl alcohol; propylene glycol; polyethylene glycol; C₉₋₁₁ or C₁₂₋₁₅ fatty alcohols; azone; alkyl pyrrolidones; diethoxy glycol (Transcutol); lecithin; etc. Surfactants can also be used as penetration enhancers.

According to an embodiment, the compositions of the invention may further comprise at least one skin benefit agent selected from the group comprising kojic acid, arbutin, deoxyarbutin, depigmenting oligopeptides, soybean extract, licorice extract, heparan sulfate and their analogs, dermatan sulfate and their analogs, chondroitin sulfate and their analogs, phyllanthus emblica extract, Bellis perennis extract, glabridin, polyphenol antioxidants, thiolic antioxidants, hydroquinone, methimazole, t-butyl hydroquinone, retinol, panthenol, Vitamin E selected from tocopherol and tocopherol acetate, Vitamin C selected from Ascorbyl Palmitate, Sodium Ascorbyl Phosphate, and Ascrobic acid, Vitamin B and derivatives thereof, moisturizing sugars selected from Xylitylglucoside, Anhydroxylitol, and Xylitol, dioic acids, corticosteroids, 4-substituted resorcinol derivatives, and mixtures thereof. In further embodiments, the at least one skin benefit agent is selected from the group consisting of Vitamin C selected from Ascorbyl Palmitate, Sodium Ascorbyl Phosphate, and Ascrobic acid, Vitamin E selected from tocopherol and tocopherol acetate, panthenol, retinol, natural extracts selected from Tasmannia Lanceolata Fruit/Leaf Extract and Hordeum Vulgare Seed Flour, and moisturizing sugars selected from Xylitylglucoside, Anhydroxylitol, and Xylitol. In further embodiments, the at least one skin benefit agent is selected from the group consisting of kojic acid, arbutin, deoxyarbutin, soybean extract, licorice extract, heparan sulfate, dermatan sulfate, chondroitin sulfate, phyllanthus emblica extract, Bellis perennis extract, glabridin, polyphenol antioxidants, thiolic antioxidants, hydroquinone, methimazole, t-butyl hydroquinone, retinol, panthenol, Vitamin E selected from tocopherol and tocopherol acetate, Vitamin C selected from Ascorbyl Palmitate, Sodium Ascorbyl Phosphate, and Ascrobic acid, Vitamin B, moisturizing sugars selected from Xylitylglucoside, Anhydroxylitol, and Xylitol, dioic acids, corticosteroids, and mixtures thereof.

According to another embodiment, the compositions of the invention may also further include liposomes (unilamellar and/or multilamellar liposomes of any size), niosome, or any encapsulation system (such as microencapsulation, nanoencapsulation, cubosome) in order to facilitate the delivery of any component(s) of the composition to its site of action. The optimal type and size of the liposome(s), the niosome(s) and/or the encapsulation system(s), and the nature of the medium in which they are dispersed, can be easily chosen by a person skilled in the art.

The compositions of the invention may be cosmetic, dermatologic or pharmaceutical compositions, and may exist in a wide variety of forms, such as emulsions, suspensions, solutions and the like. In certain embodiments, the compositions of the invention are in the form of lotions, creams, gels, solutions, sprays, cleansers, powders, ointments, waxes, lipsticks, soaps, shampoos, hydroalcoholic solutions, suspensions, scrubs, saturated pads, skin conditioning agents, and other types of cosmetic compositions. In further embodiments, the compositions of the invention may be, for example anhydrous preparations, oil-free preparations, emulsions or microemulsions of the type water-in-oil (W/O) or of the type oil-in-water (O/W), multiple emulsions, for example of the type water-in-oil-in-water (W/O/W), solid sticks, or even aerosols.

The preferred forms of the compositions of the invention are gels, lotions, emulsions, powder forms.

In some embodiments, the present invention relates to topical compositions that promote immediate skin depigmentation through the concerted action of active ingredients cysteamine or a salt thereof, azabenzene-4-carboxamide or a salt thereof and glycolic acid.

In some embodiments, the present invention relates to topical compositions that promote anti-inflammatory effect through the concerted action of active ingredients cysteamine or a salt thereof, azabenzene-4-carboxamide or a salt thereof and glycolic acid.

In some embodiments, the present invention relates to topical compositions that promote increase shelf-life through the concerted action of active ingredients cysteamine or a salt thereof, azabenzene-4-carboxamide or a salt thereof and glycolic acid.

The compositions of the invention have significantly reduced or eliminated unpleasant odour and enhanced organoleptic properties, which is more acceptable and more suitable for topical pharmaceutical and cosmetic applications.

According to an aspect of the invention, the compositions of the invention are used for depigmentation (whitening or lightening) of the human skin. According to a further aspect, the invention provides a method for inhibiting, reducing or preventing skin pigmentation. Said human skin is preferably at least one of facial skin, skin on the neck, skin on the arms, skin on the hands, skin on the legs, skin on the elbows, skin on the knees, skin on the axillary area, skin on the scalp, and skin on the male or female genital areas.

In some embodiments the compositions of the invention are used for cosmetic depigmentation of human natural healthy skin, i.e. non-therapeutic use, that is not related to treatment of diseases or pathological states of the skin. In some embodiments, cosmetic depigmentation (cosmetic inhibiting, reducing or preventing skin pigmentation) relates to skin lightening, skin radiance, skin brightening, skin evenness, skin depigmentation, anti-brown spots and/or patches. In other embodiments the skin pigmentation to be reduced or prevented according to the method of the invention can be a normal amount of pigmentation. For example, the method can be used if a subject desires to reduce or prevent pigmentation of at least a region of skin for cosmetic reasons.

Thus according to an embodiment, the invention provides the cosmetic use of the compositions of the invention for inhibiting, reducing or preventing skin pigmentation of normal healthy skin.

Said skin is preferably at least one of facial skin, skin on the neck, skin on the arms, skin on the hands, skin on the legs, skin on the elbows, skin on the knees, skin on the axillary area, skin on the scalp, and skin on the male or female genital areas. The term “normal skin” is referred to healthy skin having no pigmentation disorders or diseases.

The invention also provides a cosmetical method for inhibiting, reducing or preventing skin pigmentation of normal healthy skin, comprising topically applying the composition of the invention to the skin of a subject in need thereof.

According to another embodiment, the invention also provides a cosmetical method for reducing pigmentation of normal healthy skin in patients with generalized vitiligo in order to reduce the contrast between the diseased and the normal healthy skin, comprising topically applying the composition of the invention to the skin. In this embodiment the subject may have a condition that results in hypopigmentation of one or more regions of the skin, or localized hypomelanosis, for example in vitiligo. Affected regions of skin have reduced or absent pigmentation, and these regions can be strikingly different from adjacent unaffected regions of skin with normal and full amounts of pigmentation. By reducing the degree of pigmentation or preventing the normal degree of pigmentation in unaffected (normal healthy) skin, the contrast in pigmentation between affected skin and unaffected regions of skin can be reduced.

The cosmetical method of the invention is repeated for a number of times sufficient to inhibit, reduce or prevent skin pigmentation of normal healthy skin, for example until achievement of desired depigmentation effect (endpoint) and/or, after achievement of desired depigmentation effect the method of the invention is repeated not every day, not every consecutive day or one to six times a week to maintain the depigmentation results or effects.

In a further embodiment of the cosmetical method of the invention, when desired depigmenting effect has been achieved, the cosmetical method of the invention for inhibiting, reducing or preventing skin pigmentation of normal healthy skin can be continued to be applied not every day, not every consecutive day or one to six times a week to maintain the depigmentation results or effects.

Another aspect of the present invention provides a cosmetical method for inhibiting, reducing or preventing pigmentation of normal healthy skin, comprising the steps of
a) topically applying to an area of the skin of a subject in need thereof a first composition comprising
0.2% to 20% w/w of cysteamine or a salt thereof, preferably 5% to 20% w/w of cysteamine or a salt thereof, or 2% to 12% w/w of cysteamine or a salt thereof, or 5% to 12% w/w of cysteamine or a salt thereof, or 0.2% to 7% w/w of cysteamine of a salt thereof, or 2% to 7% w/w of cysteamine or a salt thereof, or 3% to 20% w/w of cysteamine or a salt thereof, or 1% to 12% w/w of cysteamine or a salt thereof, or 3% to 12% w/w of cysteamine or a salt thereof, or 0.2% to 7% w/w of cysteamine of a salt thereof, or 1% to 7% w/w of cysteamine or a salt thereof, and
2% to 20% w/w of glycolic acid, preferably 3.5% to 20% w/w of glycolic acid, or 3.5% to 15% w/w of glycolic acid, or 2% to 10% w/w of glycolic acid, or 2% to 15% w/w of glycolic acid,
b) rinsing off the first composition,
c) topically applying to said area of the skin a second composition comprising 0.2% to 20% w/w of an azabenzene-4-carboxamide or a salt thereof, preferably 3% to 20% of an azabenzene-4-carboxamide or a salt thereof, or 2% to 10% w/w azabenzene-4-carboxamide or a salt thereof, or 3% to 10% w/w of azabenzene-4-carboxamide or a salt thereof, or 0.2% to 10% w/w of azabenzene-4-carboxamide or a salt thereof, or 0.5% to 10% w/w of azabenzene-4-carboxamide or a salt thereof,
d) repeating the steps a), b) and c) to obtain desired skin depigmentation effect (endpoint).

In some embodiments of the cosmetical method, the first composition is topically applied in step a) for a certain amount of time, such as 15 minutes, 20 minutes, 30 minutes, 45 minutes, 1 hour, or 2 hours, to an area of the skin of a subject in need thereof, or 15 to 30 minutes, 15 to 45 minutes, 15 minutes to 1 hour, 15 minutes to 2 hours to an area of the skin of a subject in need thereof.

In other embodiments of the cosmetical method, rinsing off the first composition in step b) is typically carried out by any suitable method before topically applying the second composition.

In some embodiments, the suitable method is selected from
rinsing with water with or without a suitable detergent,
removing with any suitable solvent or cleanser,
removing with a dry or wet pad, and/or
combination thereof

The cosmetical method of the invention is repeated for a time period or number of times sufficient to obtain the desired skin depigmentation effect (endpoint), such as one week, two weeks, three weeks, four weeks, five weeks, six weeks, seven weeks, eight weeks, 2 months, three months, one year, two years, three years, or more; or such as one to four weeks, one to eight weeks, one week to one year, one week to two years, one week to five years. In another embodiment, after achievement of desired depigmentation effect, the cosmetical method of the invention is repeated not every day, not every consecutive day or one to four times a week to maintain the depigmentation results or effects.

In a further embodiment of the cosmetical method, when desired depigmentation effect (endpoint) has been achieved, the cosmetical method can be continued to be applied not every day, not every consecutive day or one to four times a week to maintain the depigmentation results or effects.

In further embodiments of the cosmetical method, the first composition consists of
0.2% to 20% w/w of cysteamine or a salt thereof, preferably 5% to 20% w/w of cysteamine or a salt thereof, or 2% to 12% w/w of cysteamine or a salt thereof, or 5% to 12% w/w of cysteamine or a salt thereof, or 0.2% to 7% w/w of cysteamine of a salt thereof, or 2% to 7% w/w of cysteamine or a salt thereof, or 3% to 20% w/w of cysteamine or a salt thereof, or 1% to 12% w/w of cysteamine or a salt thereof, or 3% to 12% w/w of cysteamine or a salt thereof, or 0.2% to 7% w/w of cysteamine of a salt thereof, or 1% to 7% w/w of cysteamine or a salt thereof,
2% to 20% w/w of glycolic acid, preferably 3.5% to 20% w/w of glycolic acid, or 3.5% to 15% w/w of glycolic acid, or 2% to 10% w/w of glycolic acid, or 2% to 15% w/w of glycolic acid,
0.01% to 1% w/w retinol, and
cosmetically acceptable excipients.

In further embodiments of the cosmetical method, the second composition consists of
0.2% to 20% w/w of azabenzene-4-carboxamide or a salt thereof, preferably 3% to 20% of an azabenzene-4-carboxamide or a salt thereof, or 2% to 10% w/w azabenzene-4-carboxamide or a salt thereof, or 3% to 10% w/w of azabenzene-4-carboxamide or a salt thereof, or 0.2% to 10% w/w of azabenzene-4-carboxamide or a salt thereof, or 0.5% to 10% w/w of azabenzene-4-carboxamide or a salt thereof,
maximum 15% w/w of skin conditioning agents selected from xylitylglucoside, anhydroxylitol, xylitol, panthenol, Tazman pepper natural extract and/or combination thereof, preferably 0.01% to 15% w/w or 0.01% to 10% of skin conditioning agents selected from xylitylglucoside, anhydroxylitol, xylitol, panthenol, Tazman pepper natural extract and/or combination thereof; preferably 0.01% to 15% or 0.01% to 10% of skin conditioning agents selected from xylitylglucoside, anhydroxylitol, xylitol, panthenol, and/or combination thereof,
0.01% to 1% retinol, and
cosmetically acceptable excipients.

In some other embodiments the compositions of the invention are used for medical/therapeutic treatment of skin hyperpigmentation disorders or diseases selected from melasma, phytophotodermatitis, lentigines (solar and senile lentigines), freckles, café-au-lait macules (which may be associated with neurofibromatosis or Albright's syndrome), acanthosis nigricans, facial dyschromia, Addison's disease, biliary cirrhosis, ectopic ACTH syndrome, eosinophilia-myalgia syndrome, folate deficiency, hemochromatosis, junctional and compound nevi, melanosis secondary to metastatic melanoma, Nelson's syndrome, pellagra, pigmented actinic keratosis, pigmented keratinocyte tumours, POEMS syndrome, porphyria cutanea tarda, seborrheic keratosis, vitamin B12 deficiency, and Whipple's disease and/or post-inflammatory hyperpigmentation; preferably skin hyperpigmentation disorders or diseases are selected from melasma, lentigines, post-inflammatory hyperpigmentation, and/or facial dyschromia. The post-inflammatory hyperpigmentation can be due to skin injury, burns, bites, diverse inflammation reactions, such as acne or lupus inflammations.

The skin hyperpigmentation disorders or diseases are typically due to increased production and accumulation of melanin, increased numbers of melanocytes and/or increased activity of melanogenic enzymes. Ultraviolet light, chronic inflammation, injury of the skin as well as abnormal α-melanocyte stimulating hormone (α-MSH) release, are triggering factors for the skin hyperpigmentation disorders or disease.

The skin hyperpigmentation can be also drug induced hyperpigmentation, light induced hyperpigmentation and chemical induced hyperpigmentation.

Conditions associated with the skin hyperpigmentation are conditions characterized at least in part by the presence of a greater-than-desired amount of endogenous skin pigmentation affecting at least a region of the skin of a subject. In one embodiment a condition associated with hyperpigmentation is a condition characterized at least in part by the presence of a greater-than-normal amount of endogenous skin pigmentation affecting at least a region of the skin of a subject. A greater-than-normal amount of endogenous skin pigmentation refers to an amount of pigmentation that is objectively greater than that amount of pigmentation present either (a) in another region of skin of the subject, including but not limited to an average amount of pigmentation of the skin of the subject, or (b) in the same region of skin of the subject at an earlier time, e.g., prior to development of the hyperpigmentation. In different embodiments the hyperpigmentation can accompany or be a manifestation of either a malignant or non-malignant (i.e., benign) condition. Endogenous skin pigmentation refers to skin pigmentation that is generated by cells in the skin, and it is to be distinguished, for example, from skin pigmentation arising from dye injected into the skin, e.g., tattooing, or other forms of exogenous skin pigmentation.

The present invention also provides the composition of the invention for use as a medicament.

The present invention also provides the composition of the invention for use in a method for inhibiting, reducing or preventing skin hyperpigmentation disorders or diseases in a subject.

The present invention further provides a method for inhibiting, reducing or preventing skin hyperpigmentation disorders or diseases, comprising topically applying the compositions of the invention to the skin of a subject in need thereof.

In a further embodiment of the method of the invention, when desired depigmenting effect has been achieved, the method of the invention for inhibiting, reducing or preventing skin hyperpigmentation disorders or diseases can be continued to be applied not every day, not every consecutive day or one to four times a week to maintain the depigmentation results or effects.

The method of the invention is repeated for a number of times sufficient to inhibit, reduce or prevent skin hyperpigmentation disorders or diseases, for example until achievement of desired depigmenting effect (endpoint) and/or, after achievement of desired depigmenting effect the method of the invention is repeated not every day, not every consecutive day or one to four times a week to maintain the depigmentation results or effects.

In the methods of the invention for inhibiting, reducing or preventing skin hyperpigmentation disorders or diseases and/or skin pigmentation of normal healthy skin, one of ordinary skill in the art will appreciate that the endpoint chosen in a particular case will vary according to the disease, condition, or disorder being treated, the skin desired appearance, the outcome desired by the patient, subject, or treating physician, and other factors. Where the composition or the combination product is being used to lighten skin colour such as, for example, in cosmetic treatment or to reverse hyperpigmentation caused by, for example, inflammation or diseases such as melasma, any one or a number of endpoints can be chosen. For example, endpoints can be defined subjectively such as, for example when the subject is simply “satisfied” with the results of the treatment. For pharmacological compositions, the endpoint can be determined by the patients or by the treating physician's satisfaction with the results of the treatment. Alternatively, endpoints can be defined objectively. For example, the patient's or subject's skin in the treated area can be compared to a colour chart. Treatment is terminated when the colour of the skin in the treated area is similar in appearance to a colour on the chart. Alternatively, the reflectance of the treated skin can be measured, and treatment (cosmetic or therapeutic) can be terminated when the treated skin attains a specified reflectance. In another method, the amount of melanin in the skin can be measured.

A hyperpigmented region of skin or a region of skin to be depigmented can involve and refer to an area (a segment) of skin from as small as about 1 mm² up to and including the entire surface of the skin. In certain common embodiments a hyperpigmented region of skin or a region of skin to be depigmented can involve and refer to a segment (an area) of skin from about 1 cm² to tens of cm². There can be a single hyperpigmented region/region to be depigmented or there can be more than one hyperpigmented region/region to be depigmented in a given subject. When there is more than one hyperpigmented region/region to be depigmented in a subject, the various hyperpigmented regions/regions to be depigmented can be similar or dissimilar to one another in size, shape, and/or pigmentation.

Thus the uses and methods of the invention for inhibiting, reducing or preventing skin hyperpigmentation disorders or diseases and/or skin pigmentation of normal healthy skin includes the step of locally (topically) administering to pigmented skin the composition of the invention or the combination product of the invention to reduce pigmentation of the skin. In one embodiment the locally administering is topically administering.

In the methods according to the invention for inhibiting, reducing or preventing skin hyperpigmentation disorders or diseases and/or skin pigmentation of normal healthy skin, the compositions or the combination products are applied to the skin, preferably human skin. The amount of the composition that is to be applied to the skin depends upon the form of the composition and its mode of application. For example, a spray formulation may be applied so as to provide a light, even coat on the skin. Similarly, lotions, creams, gels, shampoos and the like are typically applied in an amount to provide a light coating to the treatment area: consistent with the application of topical pharmaceutical ointments, creams, lotions, and the like. Generally, the rate of application, especially where all or substantially all of the skin surface, including hairy or non-hairy skin, is to be treated, is about 20 to 60 ml for the entire body, i.e., for the exposed skin of an “average individual” wearing a swimsuit and standing 1.65 m tall, weighing 68 kg, and having a 0.81 m waist. This translates to an application rate of about 2 mg/cm² of skin surface, including hairy or non-hairy skin surface. On the face, a typical application rate is 0.5 to 1.0 ml. At such levels of application, the amount of composition applied lies in the range of from about 0.1 to about 10 mg/cm², preferably from about 1 to about 3 mg/cm², of skin.

The compositions of the invention or the combination product of the invention may be applied once or more times per day depending on the activities the particular subject is engaged in. For example, a subject engaging in normal workday activities may wish to apply the compositions or the combination products twice a day, once in the morning, and once in the evening, in conjunction with normal grooming. On the other hand, if the subject plans outdoor activities such as sunbathing and athletics, the compositions or the combination products may be applied prior to, and during, such activities, much like a sunscreen composition is applied periodically during the day. The compositions or the combination products may be used for hyperpigmentation on the face and neck, or to alter the dark normal colour of the scalp or body to a lighter colour by applying appropriate compositions to the scalp, face and neck areas. However, the compositions or the combination products of the present invention may also be applied to the entire body, particularly areas which are not covered by clothing, such as the arms, neck, and lower legs.

For administration, the cosmetic, dermatological and pharmaceutical compositions of the invention may be applied to the skin (to body surface) in adequate depigmentation effective amount in the manner conventional for cosmetics and pharmaceutics, and which has a topical effect, i.e. local effect contrasting with systemic effects.

Another aspect of the present invention provides a method of reducing the unpleasant odour of cysteamine or a salt thereof, comprising combining 0.2% to 20% w/w cysteamine or a salt thereof with 0.2% to 20% w/w azabenzene-4-carboxamide or a salt thereof and 2% to 20% w/w glycolic acid.

In some embodiments of the method of reducing the unpleasant odour, the content of cysteamine or a salt thereof is 5% to 20% w/w, or 2% to 12% w/w, or 5% to 12% w/w, or 0.2% to 7% w/w, or 2% to 7% w/w or 3% to 20% w/w of cysteamine or a salt thereof, or 1% to 12% w/w of cysteamine or a salt thereof, or 3% to 12% w/w of cysteamine or a salt thereof, or 0.2% to 7% w/w of cysteamine of a salt thereof, or 1% to 7% w/w of cysteamine or a salt thereof.

In other embodiments of the method of reducing the unpleasant odour, the content of azabenzene-4-carboxamide or a salt thereof is 3% to 20% w/w, or 2% to 10% w/w, or 3% to 10% w/w, or 0.2% to 10% w/w.

In further embodiments of the method of reducing the unpleasant odour, the content of glycolic acid is 3.5% to 20% w/w, or 3.5% to 15% w/w, or 2% to 10% w/w, or 2% to 15% w/w.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

The foregoing description will be more fully understood with reference to the following Examples. Such Examples, are, however, exemplary of methods of practising the present invention and are not intended to limit the application and the scope of the invention.

### EXAMPLES

### Examples of the Compositions of the Invention

### Composition A (Emulsion E/W OSP)

Cysteamine 8%
Sodium ascorbyl phosphate 1%
Ascorbyl Palmitate 1.5%
Azabenzene-4-carboxamide 5%
glycolic acid 5%
Fragrance 0.1%

### Example 1

The topical composition comprising cysteamine or salt thereof and azabenzene-4-carboxamide at high percentages can be considered as irritant for the skin. The addition of glycolic acid is reducing the irritation, leading to a soothing effect, which is surprising as glycolic acid is usually irritating the skin. When glycolic acid is replaced by other alpha-hydroxy acid (AHA), such as lactic acid or tartaric acid or when glycolic acid is reduced to 1%, the reduction of the irritation and the soothing effect are not present.

### Tested Composition:

Cysteamine hydrochloride: 5%-20% w/w
Azabenzene-4-carboxamide: 3%-20% w/w
Glycolic acid: 3.5%-20% w/w

Test 1: Irritating potential score measure after 48-hour patch-test under occlusive conditions, performed on 20 volunteers. The results are shown below.

| | Skin | |
|---|---|---|
| | irritation | |
| Composition comprising: | score | Skin irritation potential |
| 5% cysteamine hydrochloride + | 1.48 | Irritant |
| 3% azabenzene-4-carboxamide | | |
| 12% cysteamine hydrochloride + | 1.57 | Irritant |
| 10% azabenzene-4-carboxamide | | |
| 20% cysteamine hydrochloride + | 1.63 | Very Irritant |
| 20% azabenzene-4-carboxamide | | |
| 1% glycolic acid | 0.65 | Moderately irritant |
| 3.5% glycolic acid | 0.80 | Moderately irritant |
| 20% glycolic acid | 1.55 | Irritant |
| 3.5% lactic acid | 0.73 | Moderately irritant |
| 20% lactic acid | 1.50 | Irritant |
| 3.5% tartaric acid | 0.75 | Moderately irritant |
| 20% tartaric acid | 1.53 | Irritant |
| 5% cysteamine hydrochloride + | 1.65 | Very Irritant |
| 3% azabenzene-4-carboxamide + | | |
| 1% glycolic acid | | |
| 5% cysteamine hydrochloride + | 0.05 | Non-Irritant |
| 3% azabenzene-4-carboxamide + | | |
| 3.5% glycolic acid | | |
| 12% cysteamine hydrochloride + | 0.00 | Non-Irritant |
| 10% azabenzene-4-carboxamide + | | |
| 3.5% glycolic acid | | |
| 20% cysteamine hydrochloride + | 0.06 | Non-Irritant |
| 20% azabenzene-4-carboxamide + | | |
| 20% glycolic acid | | |
| 12% cysteamine hydrochloride + | 1.55 | Irritant |
| 10% azabenzene-4-carboxamide + | | |
| 3.5% lactic acid | | |
| 12% cysteamine hydrochloride + | 1.62 | Very Irritant |
| 10% azabenzene-4-carboxamide + | | |
| 20% lactic acid | | |
| 12% cysteamine hydrochloride + | 1.56 | Irritant |
| 10% azabenzene-4-carboxamide + | | |
| 3.5% tartaric acid | | |
| 12% cysteamine hydrochloride + | 1.62 | Very Irritant |
| 10% azabenzene-4-carboxamide + | | |
| 20% tartaric acid | | |
| 20% cysteamine hydrochloride + | 1.65 | Irritant |
| 25% azabenzene-4-carboxamide + | | |
| 20% glycolic acid | | |

Test 2: measure of the haemoglobin content to evaluate the reduction of the inflammation after an exposure to SLS for 24 h.

The test was performed on 20 volunteers. On the back of each volunteer, 15 skin squares were identified. An occlusive SLS patch was applied for 24 h resulting in increase of haemoglobin content (symptoms of inflammation, such as redness). On a daily basis and for 3 days, an external blinded and trained person applied a small amount of the cream (about 50 mg) on the corresponding square with disposable latex finger pads, changing between each square. The position of each cream on each square was randomized by an external person and blinded to the volunteer and the investigator. Hemoglobin content was measured after 72 h, the results are shown below.

| | Haemoglobin content reduction |
|---|---|
| Composition comprising: | versus untreated zone |
| 5% cysteamine hydrochloride + | +0.5%^{#} |
| 3% azabenzene-4-carboxamide | |
| 12% cysteamine hydrochloride + | +1%^{#} |
| 10% azabenzene-4-carboxamide | |
| 20% cysteamine hydrochloride + | +5%^{#} |
| 20% azabenzene-4-carboxamide | |
| 1% glycolic acid | +2.1%^{#} |
| 3.5% glycolic acid | +2.3%^{#} |
| 20% glycolic acid | +3%^{#} |
| 5% cysteamine hydrochloride + | +4.6%^{#} |
| 3% azabenzene-4-carboxamide + | |
| 1% glycolic acid | |
| 5% cysteamine hydrochloride + | −24%* |
| 3% azabenzene-4-carboxamide + | |
| 3.5% glycolic acid | |
| 12% cysteamine hydrochloride + | −26%* |
| 10% azabenzene-4-carboxamide + | |
| 3.5% glycolic acid | |
| 20% cysteamine hydrochloride + | −29%* |
| 20% azabenzene-4-carboxamide + | |
| 20% glycolic acid | |
| 12% cysteamine hydrochloride + | +4.9%^{#} |
| 10% azabenzene-4-carboxamide + | |
| 3.5% lactic acid | |
| 12% cysteamine hydrochloride + | +8.0%^{#} |
| 10% azabenzene-4-carboxamide + | |
| 20% lactic acid | |
| 12% cysteamine hydrochloride + | +3.7%^{#} |
| 10% azabenzene-4-carboxamide + | |
| 3.5% tartaric acid | |
| 12% cysteamine hydrochloride + | +4.3%^{#} |
| 10% azabenzene-4-carboxamide + | |
| 20% tartaric acid | |
| 20% cysteamine hydrochloride + | +8.2%^{#} |
| 25% azabenzene-4-carboxamide + | |
| 20% glycolic acid | |

^{#}significatively damage skin barrier/induce inflammation versus untreated zone
*significatively improve skin barrier/reduce inflammation versus untreated zone

### Example 2

Cysteamine with glycolic acid has a limited depigmenting effect after 8 weeks of treatment. The addition of azabenzene-4-carboxamide is increasing synergistically the depigmenting effect and makes it significantly more visible and perceptible by the treated subjects, even at low percentage (%). When azabenzene-4-carboxamide is replaced by other azabenzene-4-carboxamide isomers, such as azabenzene-2-carboxamide and azabenzene-3-carboxamide, the synergy is not present and the depigmenting efficacy is perceived as low. Surprisingly, it has been observed that the skin depigmenting effect is improved when azabenzene-4-carboxamide is applied in a dual step method consisting of applying for a certain amount of time (such as 15 minutes) a first composition comprising cysteamine or a salt thereof and glycolic acid, then rinse this first composition off, and finally apply a separated second composition comprising azabenzene-4-carboxamide. Again, when azabenzene-4-carboxamide is replaced by other azabenzene-4-carboxamide isomers, such as azabenzene-3-carboxamide, the dual step method of application is not improving the skin depigmenting efficacy.

### Tested Composition:

Cysteamine hydrochloride: 0.2%-7% w/w
Glycolic acid: 2%-10% w/w
Azabenzene-4-carboxamide: 0.2%-10% w/w

Test 3: Measure of the skin colour index after 8 weeks of treatment (Dermacatch). The test was performed on 6 volunteers. On both arms of each volunteer, 20 skin squares were identified by UV irradiation. For 8 weeks, the volunteers were asked to apply a small amount of cream (about 50 mg) on the corresponding square with disposable latex finger pads, changing between each square. The position of each cream on each square was randomized by an external person and blinded to the volunteer and the investigator. After 8 weeks of daily application, an external investigator measured the melanin index with a Dermacatch, taking 6 measurements on each square. The results below are the average values of all the values obtained by formula (6 volunteers, 6 measurements). In addition, it was asked to the volunteers to evaluate on a range of 0-5 the efficacy of the depigmenting treatment (0=absence of efficacy; 5=very high efficacy)

| | Dermacatch Melanin Index | |
|---|---|---|
| Composition comprising: | reduction versus baseline | Subject assessment |
| 0.2% cysteamine hydrochloride + | −4% | 1- very low |
| 2% glycolic acid | | depigmenting efficacy |
| 7% cysteamine hydrochloride + | −10%* | 2- low depigmenting |
| 10% glycolic acid | | efficacy |
| 0.2% azabenzene-4-carboxamide | −6% | 1- very low |
| | | depigmenting efficacy |
| 10% azabenzene-4-carboxamide | −9%* | 2- low depigmenting |
| | | efficacy |
| 0.2% azabenzene-3-carboxamide | −5% | 1- very low |
| (isomer of azabenzene-4- | | depigmenting efficacy |
| carbomoxamide) | | |
| 10% azabenzene-3-carboxamide | −8%* | 2- low depigmenting |
| (isomer of azabenzene-4- | | efficacy |
| carbomoxamide) | | |
| 0.2% azabenzene-3-carboxamide | −4% | 1- very low |
| (isomer of azabenzene-2- | | depigmenting efficacy |
| carbomoxamide) | | |
| 10% azabenzene-3-carboxamide | −8%* | 2- low depigmenting |
| (isomer of azabenzene-2- | | efficacy |
| carbomoxamide) | | |
| 7% cysteamine hydrochloride + | −40%** | 5**- very high |
| 10% glycolic acid + | | depigmenting efficacy |
| 0.2% azabenzene-4-carboxamide | | |
| “2-step” method: | −60%*** | 5**- very high |
| 1) 7% cysteamine hydrochloride + | | depigmenting efficacy |
| 10% glycolic acid (step a, applied | | |
| for 15 min before rinsing off) | | |
| 2) 0.2% azabenzene-4-carboxamide | | |
| (step b, applied after rinsing off) | | |
| 7% cysteamine hydrochloride + | −53%** | 5**- very high |
| 10% glycolic acid + | | depigmenting efficacy |
| 10% azabenzene-4-carboxamide | | |
| “2-step” method: | −75%*** | 5**- very high |
| 1) 7% cysteamine hydrochloride + | | depigmenting efficacy |
| 10% glycolic acid (step a, applied | | |
| for 15 min before rinsing off) | | |
| 2) 10% azabenzene-4-carboxamide | | |
| (step b, applied after rinsing off) | | |
| 0.2% cysteamine hydrochloride + | −42%** | 4**- high |
| 2% glycolic acid + | | depigmenting efficacy |
| 0.2% azabenzene-4-carboxamide | | |
| “2-step” method: | −55%*** | 5**- very high |
| 1) 0.2% cysteamine hydrochloride + | | depigmenting efficacy |
| 2% glycolic acid (step a, applied | | |
| for 15 min before rinsing off) | | |
| 2) 0.2% azabenzene-4-carboxamide | | |
| (step b, applied after rinsing off) | | |
| 7% cysteamine hydrochloride + | −10%* | 2- low depigmenting |
| 10% glycolic acid + other isomers of | | efficacy |
| azenbenzene-4-carbomoximide, such | | |
| as azabenzene-3-carboxamide 10% | | |
| “2-step” method: | −9%* | 2- low depigmenting |
| 1) 7% cysteamine hydrochloride + | | efficacy |
| 10% glycolic acid (step a, applied | | |
| for 15 min before rinsing off) | | |
| 2) other isomers of azenbenzene-4- | | |
| carbomoximide, such as azabenzene- | | |
| 3-carboxamide 10% (step b, applied | | |
| after rinsing off) | | |
| 0.2% cysteamine hydrochloride + | −4% | 1- very low |
| 2% glycolic acid + other isomers of | | depigmenting efficacy |
| azenbenzene-4-carbomoximide, such | | |
| as azabenzene-3-carboxamide 0.2% | | |
| 7% cysteamine hydrochloride + | −8* | 2- low depigmenting |
| 10% glycolic acid + other isomers of | | efficacy |
| azenbenzene-4-carbomoximide, such | | |
| as azabenzene-3-carboxamide 0.2% | | |
| 7% cysteamine hydrochloride + | −9* | 2- low depigmenting |
| 10% glycolic acid + other isomers of | | efficacy |
| azenbenzene-4-carbomoximide, such | | |
| as azabenzene-2-carboxamide 10% | | |
| 7% cysteamine hydrochloride + | −7* | 2- low depigmenting |
| 10% glycolic acid + other isomers of | | efficacy |
| azenbenzene-4-carbomoximide, such | | |
| as azabenzene-2-carboxamide 0.2% | | |

*significative versus baseline
**synergistic effect of the combination
***significantly more effective than the application of the 3 ingredients applied at the same time

### Example 3

The combination of cysteamine, azabenzene-4-carboxamide and glycolic acid has a synergistic immediate depigmenting effect. In addition, when the 3 compounds are combined together, shelf-life of the composition is synergistically increased.

### Tested Composition:

Cysteamine hydrochloride: 0.2%-20% w/w
Azabenzene-4-carboxamide: 0.2%-20% w/w
Glycolic acid: 2%-20% w/w

Test 4: Measure of the skin colour index (Dermacatch) 15 seconds after the application of the topical composition. The test was performed on 6 volunteers. On both arms of each volunteer, 21 skin squares were identified. The position of each cream on each square was randomized by an external person and blinded to the volunteer and the investigator. An external blinded and trained person applied a small amount of the cream (about 50 mg) on the corresponding square with disposable latex finger pads, changing between each square and measure the skin colour index by Dermacatch, taking 6 measurements on each square. The results below are the average values of all the values obtained by formula (6 volunteers, 6 measurements). In addition, it was asked to the volunteers to evaluate on a range of 0-5 (0=absent; 5=very high) the efficacy of the immediate depigmenting treatment by comparing with the normal skin around the identified skin square.

| | Dermacatch Melanin Index | |
|---|---|---|
| Composition comprising: | reduction versus baseline | Subject assessment |
| 0.2% cysteamine hydrochloride | −0.1% | 0- absence of |
| | | immediate |
| | | depigmenting efficacy |
| 7% cysteamine hydrochloride | −0.8% | 1- very low immediate |
| | | depigmenting efficacy |
| 20% cysteamine hydrochloride | −2.1% | 1- very low immediate |
| | | depigmenting efficacy |
| 0.2% azabenzene-4-carboxamide | +0.1% | 0- absence of |
| | | immediate |
| | | depigmenting efficacy |
| 3% azabenzene-4-carboxamide | −0.5% | 1- very low immediate |
| | | depigmenting efficacy |
| 20% azabenzene-4-carboxamide | −1.3% | 1- very low immediate |
| | | depigmenting efficacy |
| 2% glycolic acid | −0.3% | 0- absence of |
| | | immediate |
| | | depigmenting efficacy |
| 20% glycolic acid | −1.5% | 1- very low immediate |
| | | depigmenting efficacy |
| 0.2% azabenzene-3-carboxamide (isomer | −0.2% | 0- absence of |
| of azabenzene-4-carboxamide) | | immediate |
| | | depigmenting efficacy |
| 20% azabenzene-3-carboxamide (isomer | −1.5% | 1- very low immediate |
| of azabenzene-4-carboxamide) | | depigmenting efficacy |
| 2% Lactic acid | −0.2% | 0- absence of |
| | | immediate |
| | | depigmenting efficacy |
| 20% Lactic acid | −1.0% | 1- very low immediate |
| | | depigmenting efficacy |
| 0.2% cysteamine hydrochloride + | −17%** | 4- high immediate |
| 0.2% azabenzene-4-carboxamide + | | depigmenting efficacy |
| 2% glycolic acid | | |
| 20% cysteamine hydrochloride + | −32%** | 4- high immediate |
| 20% azabenzene-4-carboxamide + | | depigmenting efficacy |
| 20% glycolic acid | | |
| 7% cysteamine hydrochloride + | −21%** | 4- high immediate |
| 3% azabenzene-4-carboxamide + | | depigmenting efficacy |
| 2% glycolic acid | | |
| 7% cysteamine hydrochloride + | −24%** | 4- high immediate |
| 20% azabenzene-4-carboxamide + | | depigmenting efficacy |
| 2% glycolic acid | | |
| 7% cysteamine hydrochloride + | −23%** | 4- high immediate |
| 3% azabenzene-4-carboxamide + | | depigmenting efficacy |
| 20% glycolic acid | | |
| 0.2% cysteamine hydrochloride + isomer | −0.2% | 0- absence of |
| of azabenzene-4-carboxamide, such as | | immediate |
| azabenzene-3-carboxamide 0.2% + | | depigmenting efficacy |
| 2% glycolic acid | | |
| 20% cysteamine hydrochloride + isomer | −1.5% | 1- very low immediate |
| of azabenzene-4-carboxamide, such as | | depigmenting efficacy |
| azabenzene-3-carboxamide 20% + | | |
| 20% glycolic acid | | |
| 0.2% cysteamine hydrochloride + | −0.2% | 0- absence of |
| 0.2% azabenzene-4-carboxamide + | | immediate |
| 2% lactic acid | | depigmenting efficacy |
| 20% cysteamine hydrochloride + | −1.3 | 1- very low immediate |
| 20% azabenzene-4-carboxamide + | | depigmenting efficacy |
| 20% lactic acid | | |

**synergistic effect of the combination

Test 5: Shelf-Life Estimation after Accelerated Stability Studies

The following compositions were stored at room temperature (20° C.-25° C.). Every 2 months, the pH, viscosity of the formula were measured and compared with baseline. The content of each ingredient of the composition was also dosed thanks to analytical methods and compared versus baseline. The shelf-life was determined when one of the parameter comprising pH, viscosity or analytical dosage was measured as outside the range [90%-110%] of the baseline value.

| | Shelf-life in |
|---|---|
| Composition comprising: | month |
| 0.2% cysteamine hydrochloride | 12 |
| 7% cysteamine hydrochloride | 6 |
| 20% cysteamine hydrochloride | 2 |
| 0.2% azabenzene-4-carboxamide | 12 |
| 3% azabenzene-4-carboxamide | 8 |
| 20% azabenzene-4-carboxamide | 4 |
| 2% glycolic acid | 24 |
| 20% glycolic acid | 24 |
| 0.2% azabenzene-3-carboxamide | 24 |
| 15% azabenzene-3-carboxamide | 12 |
| 2% lactic acid | 24 |
| 15% lactic acid | 24 |
| 0.2% cysteamine hydrochloride + | 60** |
| 0.2% azabenzene-4-carboxamide + | |
| 2% glycolic acid | |
| 20% cysteamine hydrochloride + | 48** |
| 15% azabenzene-4-carboxamide + | |
| 20% glycolic acid | |
| 7% cysteamine + | 54** |
| 3% azabenzene-4-carboxamide + | |
| 2% glycolic acid | |
| 7% cysteamine hydrochloride + | 54** |
| 15% azabenzene-4-carboxamide + | |
| 2% glycolic acid | |
| 7% cysteamine hydrochloride + | 60** |
| 3% azabenzene-4-carboxamide + | |
| 20% glycolic acid | |
| 0.2% cysteamine hydrochloride + isomer | 12 |
| of azabenzene-4-carboxamide such as | |
| azabenzene-3-carboxamide 0.2% + | |
| 2% glycolic acid | |
| 20% cysteamine hydrochloride + isomer | 2 |
| of azabenzene-4-carboxamide such as | |
| azabenzene-3-carboxamide 15% + | |
| 20% glycolic acid | |
| 0.2% cysteamine hydrochloride + | 12 |
| 0.2% azabenzene-4-carboxamide + | |
| 2% lactic acid | |
| 20% cysteamine hydrochloride + | 2 |
| 15% azabenzene-4-carboxamide + | |
| 20% lactic acid | |

**synergistic effect of the combination

### Example 4

The addition of 0.2% to 15% w/w of azabenzene-4-carboxamide or a salt thereof into a composition already comprising cysteamine or a salt thereof and glycolic acid provides an even bigger reduction of the unpleasant odour of cysteamine, leading to the increase of the acceptability of the product by the treated subjects.

### Tested Composition:

Cysteamine hydrochloride: 1%-20% w/w
Azabenzene-4-carboxamide: 0.2%-15% w/w
Glycolic acid: 2%-20% w/w

Test 6: Compositions were prepared in containers of 50 ml by an external operator. Perfume paper test strips were then soaked with 0.3 ml of the reference or tested composition, and then left to dry for 15 min. Then 6 volunteers were asked to sniff first the perfume paper tests strip soaked with the composition to test and then sniff reference perfume paper tests strip soaked with the reference composition (comprising the same percentage of cysteamine hydrochloride as the composition to be tested) and assign the following evaluations:
1—Unpleasant odor (such as sulfur odor) not reduced
2—Unpleasant odor (such as sulfur odor) slightly reduced
3—Unpleasant odor (such as sulfur odor) reduced
4—Unpleasant odor (such as sulfur odor) strongly reduced
5—Unpleasant odor (such as sulfur odor) totally reduced

For each preparation (baseline & tested composition), the 6 volunteers were asked to answer to the question “Is the product acceptable as a cosmetic product?” by rating it on a scale from 1 to 5 (1=not acceptable; 5=perfect).

| | | Product acceptability |
|---|---|---|
| Composition comprising: | Sniff test | as a cosmetic |
| 1% cysteamine hydrochloride | Unpleasant odor | 1- not acceptable |
| (reference container) | (baseline sniff test) | |
| 7% cysteamine hydrochloride | Very unpleasant odor | 1- not acceptable |
| (reference container) | (baseline sniff test) | |
| 20% cysteamine hydrochloride | Very unpleasant odor | 1- not acceptable |
| (reference container) | (baseline sniff test) | |
| 1% cysteamine hydrochloride + | 2- unpleasant odor | 2- barely acceptable |
| 2% glycolic acid | slightly reduced | |
| 7% cysteamine hydrochloride + | 2- unpleasant odor | 2- barely acceptable |
| 10% glycolic acid | slightly reduced | |
| 20% cysteamine hydrochloride + | 2- unpleasant odor | 2- barely acceptable |
| 20% glycolic acid | slightly reduced | |
| 1% cysteamine hydrochloride + | 4**- unpleasant odor | 4**- almost perfect |
| 2% glycolic acid + | strongly reduced | |
| 0.2% azabenzene-4-carboxamide | | |
| 7% cysteamine hydrochloride + | 4**- unpleasant odor | 4**- almost perfect |
| 10% glycolic acid + | strongly reduced | |
| 3% azabenzene-4-carboxamide | | |
| 20% cysteamine hydrochloride + | 4**- unpleasant odor | 4**- almost perfect |
| 20% glycolic acid + | strongly reduced | |
| 15% azabenzene-4-carboxamide | | |
| 7% cysteamine hydrochloride + | 2- unpleasant odor | 2- barely acceptable |
| 10% glycolic acid + isomer of | slightly reduced | |
| azabenzene-4-carboxamide, such | | |
| as azabenzene-3-carboxamide 3% | | |
| 20% cysteamine hydrochloride + | 3- unpleasant odor | 2- barely acceptable |
| 20% glycolic acid + isomer of | reduced | |
| azabenzene-4-carboxamide, such | | |
| as azabenzene-3-carboxamide 15% | | |
| 7% cysteamine hydrochloride + | 3- unpleasant odor | 2- barely acceptable |
| 10% glycolic acid + isomer of | reduced | |
| azabenzene-4-carboxamide, such | | |
| as azabenzene-2-carboxamide 3% | | |
| 20% cysteamine hydrochloride + | 3- unpleasant odor | 2- barely acceptable |
| 20% glycolic acid + isomer of | reduced | |
| azabenzene-4-carboxamide, such | | |
| as azabenzene-2-carboxamide 15% | | |

**synergistic effect of the combination

1. A composition comprising:
0.2% to 20% w/w of cysteamine or a salt thereof,
0.2% to 20% w/w of azabenzene-4-carboxamide or a salt thereof, and
2% to 20% w/w of glycolic acid.

2. The composition of claim 1, comprising 5% to 20% w/w of cysteamine or a salt thereof, or 2% to 12% w/w of cysteamine or a salt thereof.

3. The composition of claim 1, comprising 3% to 20% w/w of azabenzene-4-carboxamide or a salt thereof.

4. The composition of claim 1, comprising 3.5% to 20% w/w of glycolic acid.

5. The composition of claim 1, comprising
5% to 20% w/w of cysteamine or a salt thereof,
3% to 20% w/w of azabenzene-4-carboxamide or a salt thereof, and
3.5% to 20% w/w of glycolic acid.

6. The composition of claim 1, comprising
0.2% to 7% w/w of cysteamine or a salt thereof,
0.2% to 10% w/w of azabenzene-4-carboxamide or a salt thereof, and
2% to 10% w/w of glycolic acid.

7. The composition of claim 1, further comprising at least one cosmetically acceptable excipient, dermatologically acceptable excipient, or pharmaceutically acceptable excipient.

8. The composition of claim 1, further comprising at least one skin benefit agent comprising kojic acid, arbutin, deoxyarbutin, a depigmenting oligopeptides, a soybean extract, a licorice extract, heparan sulfate or an analog thereof, dermatan sulfate or an analog thereof, chondroitin sulfate or an analog thereof, a phyllanthus emblica extract, a Bellis perennis extract, glabridin, a polyphenol antioxidants, a thiolic antioxidants, hydroquinone, methimazole, t-butyl hydroquinone, retinol, panthenol, Vitamin E selected from tocopherol and tocopherol acetate, Vitamin C selected from Ascorbyl Palmitate, Sodium Ascorbyl Phosphate, and Ascrobic acid, Vitamin B or derivatives thereof, a moisturizing sugar selected from Xylitylglucoside, Anhydroxylitol, and Xylitol, a dioic acids, a corticosteroids, a 4-substituted resorcinol derivatives, or a mixture thereof.

9. The composition of claim 1, wherein said composition is in the form of a lotion, a cream, a gel, a solution, a spray, a cleanser, a powder, an ointment, a wax, a lipstick, a soap, a shampoo, a hydroalcoholic solution, a suspension, a scrub, a saturated pad, or a skin conditioning agent.

10. A method for inhibiting, reducing, or preventing a skin hyperpigmentation disease or disorders in a subject in need thereof, the method comprising administering the composition of claim 1 to the subject.

11. The method of claim 10, wherein said hyperpigmentation diseases or disorder comprises melasma, phytophotodermatitis, lentigines, freckles, café-au-lait macules, acanthosis nigricans, facial dyschromia, Addison's disease, biliary cirrhosis, ectopic ACTH syndrome, eosinophilia-myalgia syndrome, folate deficiency, hemochromatosis, junctional and compound nevi, melanosis secondary to metastatic melanoma, Nelson's syndrome, pellagra, pigmented actinic keratosis, pigmented keratinocyte tumours, POEMS syndrome, porphyria cutanea tarda, seborrheic keratosis, vitamin B12 deficiency, Whipple's disease, or post-inflammatory hyperpigmentation.

12. A cosmetic method for inhibiting, reducing, or preventing pigmentation of normal healthy skin, the method comprising topically applying the composition of claim 1 to the skin of a subject in need thereof.

13. A cosmetic method for inhibiting, reducing, or preventing pigmentation of normal healthy skin, the method comprising the steps of:
a) topically applying to an area of the skin of a subject in need thereof a first composition comprising:
0.2% to 20% w/w of cysteamine or a salt thereof, and
2% to 20% w/w of glycolic acid,
b) rinsing off the first composition,
c) topically applying to said area of the skin a second composition comprising 0.2% to 20% w/w of an azabenzene-4-carboxamide or a salt thereof,
d) repeating the steps a), b) and c) to obtain desired skin depigmentation effect.

14. A combination product comprising a first composition and a second composition for separate topical application, wherein:
the first composition comprises 0.2% to 20% w/w of cysteamine or a salt thereof and 2% to 20% w/w of glycolic acid,
and
the second composition comprises 0.2% to 20% w/w of azabenzene-4-carboxamide or a salt thereof.

15. The combination product of claim 14 consisting of:
the first composition consisting of;
0.2% to 20% w/w of cysteamine or a salt thereof,
2% to 20% w/w of glycolic acid,
0.01% to 1% w/w retinol, and
cosmetically acceptable excipients,
and
the second composition consisting of:
0.2% to 20% w/w of azabenzene-4-carboxamide or a salt thereof,
maximum 15% w/w of skin conditioning agents selected from xylitylglucoside, anhydroxylitol, xylitol, panthenol, Tazman pepper natural extract, and a combination thereof,
0.01% to 1% retinol, and
cosmetically acceptable excipients.

16. A method of reducing the unpleasant odour of cysteamine or a salt thereof, comprising combining 0.2% to 20% w/w cysteamine or a salt thereof with 0.2% to 20% w/w azabenzene-4-carboxamide or a salt thereof and 2% to 20% w/w glycolic acid.
